(19) United States
(12) Patent Application Publication
(10) Pub. No.: US 2004/0137379 A1
(43) Pub. Date: Jul. 15, 2004

(54) Photoresist stripping agent

(76) Inventor: Kazuto Ikemoto, Niigata (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/750,822

(22) Filed: Jan. 5, 2004

(30) Foreign Application Priority Data
Jan. 9, 2003 (JP) 003700/2003

(51) Int. Cl. G03F 7/32; C07C 45/00

(52) U.S. Cl. 430/331; 568/458; 430/256

(57) ABSTRACT

The photoresist stripping agent of the present invention contains a reaction product that is produced by the reaction of formaldehyde and an alkanol amine in a molar ratio of 0.8 or less. The photoresist stripping agent easily removes, at low temperatures in a short period of time, photoresist layers applied on substrates, photoresist layers remaining after etching and photoresist residues after ashing subsequent to etching. The photoresist stripping agent also removes the photoresist layers and photoresist residues without corroding substrates, wiring materials, insulating layers, etc. to enable the fine processing and provide high precision circuits.

### BACKGROUND OF THE INVENTION

1. Field of the Invention

Photoresists have been used in the lithographic production of wide range of devices including integrated circuits such as IC and LSI, display devices such as LCD and EL device, printed boards, micro machines, DNA chips and micro plants. The present invention relates, particularly, to a photoresist stripping agent for removing photoresists from various substrates carrying the photoresists.

2. Description of the Prior Art

In conventional techniques, photoresists are removed by alkaline stripping agents. However, the photoresist stripping ability of known alkaline stripping agents is insufficient for recently developed fine process and short-time treatment in the production of semiconductor devices and liquid crystal display panels. Therefore, it has been demanded to further improve the stripping ability. A resist stripping agent containing hydroxylamine is proposed. However, hydroxylamine is easy to be decomposed. To solve the above problems, a resist stripping agent containing a compound having a methylol amine structure has been developed (for example, Japanese Patent Application Laid-Open No. 2000-250230). However, there still remains a demand for a further improved resist stripping ability.

Various materials are used in the recent production of semiconductor devices for semiconductor integrated circuits and liquid crystal display devices. Therefore, it is required to develop a photoresist stripping agent that is free from corrosion to these materials and various substrates.

In addition, the photoresist stripping ability of known photoresist stripping agents is lowered during a long-term use because of the absorption of carbon dioxide gas in air.

### SUMMARY OF THE INVENTION

An object of the present invention is to solve the above problems on the known photoresist stripping agents and to provide a photoresist stripping agent that is capable of easily removing, at low temperatures in a short period of time, photoresist layers applied on substrates, photoresist layers remaining after etching and photoresist residues after ashing subsequent to etching. Another object of the present invention is to provide a photoresist stripping agent that is capable of removing photoresist layers and photoresist residues without corroding the substrates, insulating layers, wiring materials, etc., thereby enabling the fine processing and producing high precision circuits. Still another object of the present invention is to provide a method for removing photoresists using the photoresist stripping composition. Still another object of the present invention is to provide a photoresist stripping agent that is little lowered in its photoresist stripping ability by the absorption of carbon dioxide gas in air.

As a result of extensive study, the inventors have found that a photoresist stripping agent containing a reaction product that is produced by the reaction of formaldehyde and an alkanolamine in a molar ratio (formaldehyde/alkanolamine) of 0.8 or less. Such a photoresist stripping agent easily removes, at low temperatures in a short period of time, photoresist layers applied on substrates, photoresist layers remaining after etching and photoresist residues after ashing subsequent to etching. The photoresist stripping agent also removes the photoresist layers and photoresist residues without corroding substrates, wiring materials and insulating layers to enable the fine processing and provide high precision circuits.

### BRIEF DESCRIPTION OF THE DRAWINGS

FIG. 1 is a chart showing ¹³C-NMR spectra of a reaction liquid of formaldehyde and monoethanolamine (aldehyde/amine=0.5 by mole). The chemical sifts of peaks attributable to the formaldehyde-monoethanolamine reaction product in the reaction liquid are found at 49.31, 61.19, 64.72 and 68.75 ppm.

### DETAILED DESCRIPTION OF THE INVENTION

The photoresist stripping agent of the present invention contains at least one formaldehyde-alkanolamine reaction product which is a product of the reaction between formaldehyde and an alkanolamine. As an example of the reaction product of an amine and an aldehyde, methylolamine has been known in the art. The photoresist stripping agent of the present invention contains, as the effective ingredient, an formaldehyde-alkanolamine reaction product other than methylolamine. The chemical structure of the formaldehyde-alkanolamine reaction product is not completely known. For example, the following chemical structures are described in Chemical Review, vol.126 (1939) p297-338, U.S. Pat. No. 5,486,605, Japanese Patent Publication No. 46-26903 and Soviet Patent No. 1534029:

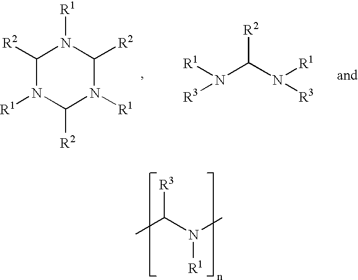

wherein R¹ and R³ are substituent groups derived from the amine, and R² is substituent group derived from the aldehyde.

The formaldehyde-alkanolamine reaction product is produced by the reaction of formaldehyde and the alkanolamine. Formalin and paraformaldehyde may be used as formaldehyde. Examples of the alkanolamines include ethanolamine, N-methylethanolamine, N-ethylethanolamine, N-propylethanolamine, N-butylethanolamine, diethanolamine, isopropanolamine, N-methylisopropanolamine, N-ethylisopropanolamine, N-propylisopropanolamine, 2-aminopropane-1-ol, N-methyl-2-amino-propane-1-ol, and N-ethyl-2-amino-propane-1-ol, with ethanolamine, N-methylethanolamine and isopropanolamine being particularly preferred.

To produce the formaldehyde-alkanolamine reaction product, the alkanolamine may be used alone or in combination of two or more. In addition, the formaldehyde-alkanolamine reaction product may be used in the form of a salt with inorganic acid or organic acid.

Preferred examples of the formaldehyde-alkanolamine reaction products include a formaldehyde-monoethanolamine condensate and a formaldehyde-isopropanolamine condensate.

The photoresist stripping capability of the formaldehyde-alkanolamine reaction product is enhanced by the co-existence of an alkali compound. Examples of the alkali compounds include alkylamines, alkanolamines, polyamines, cyclic amines, quaternary ammonium compounds and hydroxylamine compounds.

Examples of the alkylamines include primary alkylamines such as methylamine, ethylamine, n-propylamine, isopropylamine, n-butylamine, sec-butylamine, isobutylamine, tert-butylamine, pentylamine, 2-aminopentane, 3-aminopentane, 1-amino-2-methylbutane, 2-amino-2-methylbutane, 3-amino-2-methylbutane, 4-amino-2-methylbutane, hexylamine, 5-amino-2-methylpentane, heptylamine, octylamine, nonylamine, decylamine, undecylamine, dodecylamine, tridecylamine, tetradecylamine, pentadecylamine, hexadecylamine, heptadecylamine, and octadecylamine; secondary alkylamines such as dimethylamine, diethylamine, dipropylamine, diisopropylamine, dibutylamine, diisobutylamine, di-sec-butylamine, di-tert-butylamine, dipentylamine, dihexylamine, diheptylamine, dioctylamine, dinonylamine, didecylamine, methylethylamine, methylpropylamine, methylisopropylamine, methylbutylamine, methylisobutylamine, methyl-sec-butylamine, methyl-tert-butylamine, methylamylamine, methylisoamylamine, ethylpropylamine, ethylisopropylamine, ethylbutylamine, ethylisobutylamine, ethyl-sec-butylamine, ethylamine, ethylisoamylamine, propylbutylamine, and propylisobutylamine; tertiary alkylamines such as trimethylamine, triethylamine, tripropylamine, tributylamine, tripentylamine, dimethylethylamine, methyldiethylamine, and methyldipropylamine.

Examples of the alkanolamines include ethanolamine, N-methylethanolamine, N-ethylethanolamine, N-propylethanolamine, N-butylethanolamine, diethanolamine, isopropanolamine, N-methylisopropanolamine, N-ethylisopropanolamine, N-propylisopropanolamine, 2-aminopropane-1-ol, N-methyl-2-amino-propane-1-ol, N-ethyl-2-amino-propane-1-ol, 1-aminopropane-3-ol, N-methyl-1-aminopropane-3-ol, N-ethyl-1-aminopropane-3-ol, 1-aminobutane-2-ol, N-methyl-1-aminobutane-2-ol, N-ethyl-1-aminobutane-2-ol, 2-aminobutane-1-ol, N-methyl-2-aminobutane-1-ol, N-ethyl-2-aminobutane-1-ol, 3-aminobutane-1-ol, N-methyl-3-aminobutane-1-ol, N-ethyl-3-aminobutane-1-ol, 1-aminobutane-4-ol, N-methyl-1-aminobutane-4-ol, N-ethyl-1-aminobutane-4-ol, 1-amino-2-methylpropane-2-ol, 2-amino-2-methylpropane-1-ol, 1-aminopentane-4-ol, 2-amino-4-methylpentane-1-ol, 2-aminohexane-1-ol, 3-aminoheptane-4-ol, 1-aminooctan-2-ol, 5-aminooctan-4-ol, 1-aminopropane-2,3-diol, 2-aminopropane-1,3-diol, tris(oxymethyl)aminomethane, 1,2-diaminopropane-3-ol, 1,3-diaminopropane-2-ol, and 2-(2-aminoethoxy)ethanol.

Examples of the polyamines include ethylenediamine, propylenediamine, trimethylenediamine, tetramethylenediamine, 1,3-diaminobutane, 2,3-diaminobutane, pentamethylenediamine, 2,4-diaminopentane, hexamethylenediamine, heptamethylenediamine, octamethylenediamine, nonamethylenediamine, N-methylethylenediamine, N,N-dimethylethylenediamine, trimethylethylenediamine, N-ethylethylenediamine, N,N-diethylethylenediamine, triethylethylenediamine, 1,2,3-triaminopropane, hydrazine, tris(2-aminoethyl)amine, tetra(aminomethyl)methane, diethylenetriamine, triethylenetetramine, tetraethylpentamine, heptaethyleneoctamine, nonaethylenedecamine, and diazabicycloundecene.

Examples of the hydroxylamine compounds include hydroxylamine, N-methylhydroxylamine, N-ethylhydroxylamine, N,N-diethylhydroxylamine, and O-methylhydroxylamine.

Examples of the cyclic amines include pyrrole, 2-methylpyrrole, 3-methylpyrrole, 2-ethylpyrrole, 3-ethylpyrrole, 2,3-dimethylpyrrole, 2,4-dimethylpyrrole, 3,4-dimethylpyrrole, 2,3,4-trimethylpyrrole, 2,3,5-trimethylpyrrole, 2-pyrroline, 3-pyrroline, pyrrolidine, 2-methylpyrrolidine, 3-methylpyrrolidine, pyrazole, imidazole, 1,2,3-triazole, 1,2,3,4-tetrazole, piperidine, 2-pipecoline, 3-pipecoline, 4-pipecoline, 2,4-lupetidine, 2,6-lupetidine, 3,5-lupetidine, piperazine, 2-methylpiperazine, 2,5-dimethylpiperazine, 2,6-methylpiperazine, and morpholine.

Examples of the quaternary ammonium compounds include tetramethylammonium hydroxide, tetraethylammonium hydroxide, tetrapropylammonium hydroxide, tetrabutylammonium hydroxide, choline hydroxide, and acetylcholine hydroxide.

Also, the formaldehyde-alkanolamine reaction product per se can serve as the alkali compound because it is an alkaline compound.

In addition to the alkali compounds recited above, other compounds may be used in the present invention without any specific limitation as far as they shows alkaline nature.

Of the above alkali compounds, preferred are methylamine, ethylamine, propylamine, butylamine, ethanolamine, N-methylethanolamine, N-ethylethanolamine, diethanolamine, isopropanolamine, 2-(2-aminoethoxy)ethanol, ethylenediamine, propylenediamine, butylenediamine, diethylenetriamine, piperazine, and morpholine.

The alkali compounds may be used alone or in combination of two or more.

To enhance the photoresist stripping capability, the photoresist stripping agent of the present invention may contain an organic solvent. The organic solvent is not specifically limited as far as it is miscible with the alkanolamine-formaldehyde reaction product. The organic solvents soluble in water are preferred. Examples thereof include ether solvents such as ethylene glycol, ethylene glycol monoethyl ether, ethylene glycol monobutyl ether, diethylene glycol monomethyl ether, diethylene glycol monoethyl ether, diethylene glycol monobutyl ether, propylene glycol monomethyl ether, propylene glycol monoethyl ether, propylene glycol monobutyl ether, dipropylene glycol monomethyl ether, dipropylene glycol monoethyl ether, dipropylene glycol monobutyl ether, ethylene glycol dimethyl ether, and dipropylene glycol dimethyl ether; amide solvents such as formamide, monomethylformamide, dimethylformamide, monoethylformamide, diethylformamide, acetamide, monomethylacetamide, dimethylacetamide, monoethylacetamide, diethylacetamide, N-methylpyrrolidone, and N-ethylpyrrolidone; alcohol solvents such as methyl alcohol, ethyl alcohol, isopropanol, ethylene glycol, and propylene glycol; sulfoxide solvents such as dimethyl sulfoxide; sulfone solvents such as dimethyl sulfone, diethyl sulfone, bis(2-hydroxy sulfone), and tetramethylene sulfone; imidazolidinone solvents such as 1,3-dimethyl-2-imidazolidinone, 1,3-diethyl-2-imidazolidinone and 1,3-diisopropyl-2-imidazolidinone; and lactone solvents such as γ-butyrolactone and δ-valerolactone.

Of the above solvents, preferred are dimethyl sulfoxide, N,N-dimethylormamide, N,N-dimethylacetamide, N-methylpyrrolidone, diethylene glycol monomethyl ether, diethylene glycol monobutyl ether, dipropylene glycol monomethyl ether, dipropylene glycol monobutyl ether, and propylene glycol, because these solvents are easily available and easy to handle because of their high boiling points.

The photoresist stripping agent of the present invention may contain an anticorrosion agent such as aromatic hydroxy compounds, sugar alcohols, triazole compounds and chelating compounds.

Examples of the aromatic hydroxy compounds include phenol, cresol, xylenol, pyrocatechol, tert-butylcatechol, resorcinol, hydroquinone, pyrogallol, 1,2,4-benzenetriol, salicyl alcohol, p-hydroxybenzyl alcohol, o-hydroxybenzyl alcohol, p-hydorxyphenethyl alcohol, p-aminophenol, m-aminophenol, diaminophenol, aminoresorcinol, p-hydroxybenzoic acid, o-hydroxybenzoic acid, 2,4-dihydroxybenzoic acid, 2,5-dihydroxybenzoic acid, 3,4-dihydroxybenzoic acid, 3,5-dihydroxybenzoic acid, and gallic acid. Examples of the sugar alcohols include sorbitol, xylitol and palatinit. Examples of the triazole compounds include benzotriaole, aminotriazole and aminotetrazole. Examples of the chelating compounds include phosphoric acid-based compounds such as 1,2-propanediaminetetramethylenephosphonic acid and hydroxyethanephosphonic acid; carboxylic acid-based compounds such as ethylenediaminetetraacetic acid, dihydroxyethylglycine, nitrilotriacetic acid, oxalic acid, citric acid, malic acid, and tartaric acid; amine compounds such as bipyridine, tetraphenylporphyrin, phenanthroline, and 2,3-pyridinediol; oxime compounds such as dimethylglyoxime and diphenylglyoxime; and acetylene compounds such as phenylacetylene and 2,5-dimethyl-3-hexyne-2,5-diol. These compounds may be used alone or in combination of two or more.

The content of the formaldiehyde-alkanolamine reaction product in the photoresist stripping agent is preferably 0.001 to 100% by weight and more preferably 0.01 to 50% by weight. The content of the alkali compound is preferably 0 to 99.999% by weight, more preferably 10 to 99.99% by weight of the photoresist stripping agent. Since the formaldehyde-alkanolamine reaction product also acts as the alkali compound, the photoresist stripping agent containing only the formaldehyde-alkanolamine reaction product exhibits an enough effect for the photoresist stripping.

The content of the organic solvent may be selected according to the viscosity and specific gravity of the photoresist stripping agent and the conditions of etching and ashing processes, and not specifically limited. Preferably the content is 0 to 99% by weight, and more preferably 10 to 99% by weight based on the photoresist stripping agent.

The content of the anticorrosion agent is not particularly limited. If used, the content of the anticorrosion agent is preferably 0.1 to 30% by weight, and more preferably 1 to 15% by weight based on the photoresist stripping agent.

The use of water is not critical in the present invention, and the content thereof may be determined according to the conditions of etching and ashing processed, etc. If used, the content of water is preferably 1 to 50% by weight, and more preferably 5 to 40% by weight based on the photoresist stripping agent.

Generally, methylolamine is obtained as an equimolar reaction product in the reaction between an amine and formaldehyde. However, the specific feature of the present invention resides in the use of the formaldehyde-alkanolamine reaction product other than methylolamine. Japanese Patent Application Laid-Open No. 2000-250350 teaches that methylolamine enhances the photoresist stripping capability. In face of this teaching, the inventors have found that the photoresist stripping capacity can be further enhanced by the formaldehyde-alkanolamine reaction product other than methylolamine and accomplished the present invention. However, it should be noted that the use of methylolamine in combination with the formaldehyde-alkanolamine reaction product is not excluded in the present invention.

Particularly effective for photoresist stripping is an formaldehyde-alkanolamine reaction product that is produced by the reaction between formaldehyde and an excessive amount of the alkanolamine. The molar ratio of formaldehyde/alkanolamine is preferably 0.8 or less, more preferably 0.001 to 0.8, and still more preferably 0.01 to 0.5.

In the present invention, the formaldehyde-alkanolamine reaction product is produced in the following manner. Into a predetermined amount of the alkanolamine, formaldehyde is slowly added so as to regulate the formaldehyde/alkanolamine molar ratio within the above range. The addition of formaldehyde is preferably completed over 30 to 1200 min under stirring while maintaining the temperature of the reaction solution at 70° C., preferably at 30 to 60° C. After completing the addition of formaldehyde, it is preferred to continue the stirring for 30 to 1200 min while maintaining the temperature of the reaction solution at 70° C., preferably at 30 to 60° C., thereby completing the reaction. The reaction is preferably conducted in an inert gas atmosphere, for example, in nitrogen gas stream. In addition, the reaction may be conducted in the absence of solvent or may be conducted in the presence of the organic solvent mentioned above. The final reaction solution may be used as the photoresist stripping agent without separating the formaldehyde-alkanolamine reaction product.

The chemical structure of the formaldehyde-alkanolamine reaction product, particularly formaldehyde-ethanolamine reaction product, produced in the above manner is characterized by at least the peaks at 45 to 50, 61 to 62 and 64 to 70 ppm of ¹³C-NMR (DMSO-d6) spectra.

The formaldehyde-alkanolamine reaction product is considered to exhibit the photoresist stripping effect in the following manner. When the photoresist stripping agent is brought into contact with photoresist, the formaldehyde-alkanolamine reaction product therein is bonded to the photoresist to increase the solubility of the photoresist, this facilitating the removal of the photoresist. It is considered that a Mannich reaction product may contribute to the photoresist stripping. The decomposition and dissolution of the photoresist are promoted by the coexistence of the alkali compound, this enhancing the photoresist stripping capability.

The photoresist removal by the photoresist stripping agent of the present invention in the production of semiconductor devices is generally carried out at room temperature to 150° C. Since the photoresist stripping agent of the present invention can remove resists at temperatures as low as 70° C. or lower, the undesirable attack to the materials of semiconductors can be effectively prevented.

The photoresist stripping agent of the present invention is applicable to the photoresist removal in the production of semiconductor devices which are made of various materials. Examples of such materials include silicon, amorphous silicon, polysilicon, silicon oxide, silicon nitride, copper, copper alloy, aluminum, aluminum alloy, gold, platinum, silver, titanium, titanium-tungsten, titanium nitride, tungsten, tantalum, tantalum compound, chromium, chromium oxide, chromium alloy, semiconductor wiring materials such as indium-tin-oxide (ITO), compound semiconductors such as gallium-arsenic, gallium-phosphorus and indium-phosphorus, dielectric materials such as strontium-bismuth-tantalum, and glass for LCD substrate.

The photoresist removal using the photoresist stripping agent of the present invention in the production of semiconductor devices is carried out, for example, by the following manner. A photoresist composition is applied on an electrically conductive layer formed on a substrate to form a photoresist layer, which is then patterned by exposure to light and development. The non-masked region of the electrically conductive layer is etched using the patterned photoresist layer as the mask. Thereafter, the etched substrate is brought into contact with the photoresist stripping agent to remove the remaining photoresist layers. If desired, the remaining photoresist layers may be subjected to ashing treatment after the etching process, and then, the photoresist residues are removed using the photoresist stripping agent. After the removal of photoresist layers or photoresist residues, the substrate may be rinsed with an organic solvent such as alcohol or water.

The present invention will be explained in more detail by reference to the following example which should not be construed to limit the scope of the present invention.

### SYNTHESIS EXAMPLE 1

Production of Formaldehyde-monoethanolamine Condensate (Aldehyde/amine=0.5 by Molar Ratio)

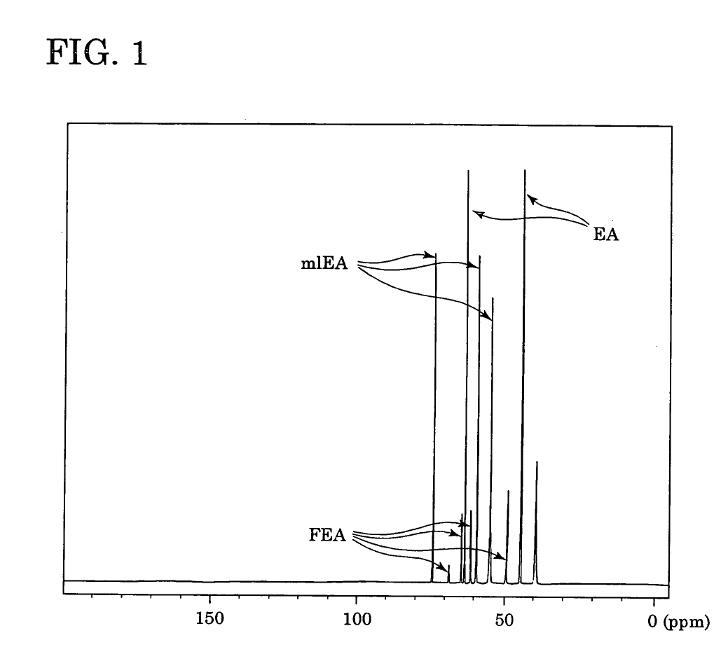

Into 61.0 g of monoethanolamine, 15 g of paraformaldehyde was slowly added under stirring while cooling the solution so as to maintain the temperature at 70° C. or lower, thereby obtaining a reaction product A in a solution form. All the procedure was conducted in nitrogen gas stream. A chart showing the ¹³C-NMR spectra (DMSO-d₆) of the reaction product A is shown in FIG. 1. In. FIG. 1, EA is monoethanolamine, m1EA is methylolethanolamine, and FEA is formaldehyde-monoethanolamine reaction product.

### SYNTHESIS EXAMPLE 2

Production of Formaldehyde-monoethanolamine Condensate (Aldehyde/amine=0.8 by Molar Ratio)

Into 61.0 g of monoethanolamine, 24 g of paraformaldehyde was slowly added under stirring while cooling the solution so as to maintain the temperature at 70° C. or lower, thereby obtaining a reaction product A in a solution form. All the procedure was conducted in nitrogen gas stream.

### EXAMPLES 1-5 and COMPARATIVE EXAMPLES 1-2

A 6-inch silicon wafer preliminarily surface-treated with a silicon compound was spin-coated with a photoresist PFR-7900. By baking at 160° C., a substrate carrying a photoresist layer of 10,000 Å thick was prepared.

The substrate thus prepared was immersed in each photoresist stripping agent listed in Table 1 at 50° C. After predetermined time intervals, each substrate was taken out of the photoresist stripping agent, rinsed with water, dried by nitrogen gas blow, and then observed under an optical microscope to determine the time required for removing the photoresist layer. The results are shown in Table 1.

TABLE 1

| | | | Formaldehyde- | | | | |
|---|---|---|---|---|---|---|---|
| | | | alkanolamine | | | | Time required |
| | Alkanolamine | | reaction product | | Solvent | | for photoresist |
| | kind | wt % | kind | wt % | kind | wt % | removal |
| Examples | | | | | | | |
| 1 | EA | 65 | Reaction | 5 | DMSO | 30 | 20 s |
| | | | product A | | | | |
| 2 | EA | 65 | Reaction | 5 | DMAC | 30 | 20 s |
| | | | product A | | | | |
| 3 | EA | 65 | Reaction | 5 | DMSO | 30 | 30 s |
| | | | product B | | | | |
| 4 | EA | 66.5 | Reaction | 3.5 | DMSO | 30 | 40 s |
| | | | product B | | | | |
| 5 | EA | 69 | Reaction | 1 | DMSO | 30 | 60 s |
| | | | product A | | | | |
| Comparative | | | | | | | |
| Examples | | | | | | | |
| 1 | EA | 70 | — | — | DMSO | 30 | 180 s |
| 2 | EA | 69 | mlEA | 1 | DMSO | 30 | 120 s |

EA: Monoethanolamine
DMAC: Dimethylacetamide
DMSO: Dimethylsulfoxide
mlEA: methylolethanolamine

### EXAMPLES 6-9 and COMPARATIVE EXAMPLES 3-4

Carbon dioxide gas was flown into a mixed solution of 68.5 g of monoethanolamine and 30 g of DMSO to dissolve 1.5 g of carbon dioxide, the resultant liquid being referred to as “carbon dioxide deterioration stripping liquid.” After adding each additive shown in Table 2 to the carbon dioxide deterioration stripping liquid, the photoresist stripping test was conducted in the same manner as in Examples 1-5. The results are shown in Table 2.

TABLE 2

| | Additives | | Time required for |
|---|---|---|---|
| | kind | wt % | photoresist removal |
| Examples | | | |
| 6 | Reaction product A | 5 | 30 s |
| 7 | Reaction product A | 2.5 | 60 s |
| 8 | Reaction product A | 1.25 | 90 s |
| 9 | Reaction product A | 5 | 30 s |
| | Citric acid | 1 | |
| Comparative Examples | | | |
| 3 | — | — | 240 s |
| 4 | Citric acid | 1 | 240 s |

### EXAMPLE 10

The production of Synthesis Example 1 was repeated except for changing paraformaldehyde to 32% formalin to prepare a formaldehyde-monoethanolamine reaction product. Using the formaldehyde-monoethanolamine reaction product thus prepared, the photoresist stripping test was conducted in the same manner as in Example 1. The photoresist layer was completely removed after 20 s of the immersion.

### EXAMPLE 11

The production of Synthesis Example 1 was repeated except for changing monoethanolamine to isopropanolamine to prepare a formaldehyde-isopropanolamine reaction product. Using the formaldehyde-isopropanolamine reaction product thus prepared, the photoresist stripping test was conducted in the same manner as in Example 1. The photoresist layer was completely removed after 20 s of the immersion.

### EXAMPLE 12

In 30 g of dimethylsulfoxide, 1 g of paraformaldehyde and 69 g of monoethanolamine (aldehyde/amine=0.03 by molar ratio) were allowed to react to produce a solution containing a formaldehyde-monoethanolamine reaction product. Using the solution thus obtained, the photoresist stripping test was conducted in the same manner as in Example 1. The photoresist layer was completely removed after 30 s of the immersion.

### EXAMPLE 13

Corrosion Test

An amorphous silicon (a-Si) substrate carrying an aluminum layer was immersed in each of the photoresist stripping agents of Examples 3-7 at 70° C. for 30 min to determine the etching rates of a-Si and Al using an optical thickness meter for a-Si and fluorescent X rays for Al. The etching rate was 5 Å/min or less for both a-Si and Al.

The photoresist stripping agent of the present invention can remove photoresist layers and photoresist residues in a short period of time without corroding substrates, wiring materials, etc. In addition, the photoresist stripping agent of the present invention is resistant to the deterioration of the photoresist stripping ability due to the absorption of carbon dioxide gas.

What is claimed is:

1. A photoresist stripping agent comprising a reaction product that is produced by the reaction of formaldehyde and an alkanolamine in a molar ratio of 0.8 or less.

2. The photoresist stripping agent according to claim 1, wherein the alkanolamine is at least one compound selected from the group consisting of ethanolamine, N-methylethanolamine, N-ethylethanolamine, N-propylethanolamine, N-butylethanolamine, diethanolamine, isopropanolamine, N-methylisopropanolamine, N-ethylisopropanolamine, N-propylisopropanolamine, 2-aminopropane-1-ol, N-methyl-2-amino-propane-1-ol, and N-ethyl-2-amino-propane-1-ol.

3. The photoresist stripping agent according to claim 1, further comprising an alkali compound.

4. The photoresist stripping agent according to claim 3, wherein the alkali compound is at least one compound selected from the group consisting of alkylamines, alkanolamines, polyamines, cyclic amines, quaternary ammonium salts and hydroxylamine compounds.

5. The photoresist stripping agent according to claim 1, further comprising an organic solvent.

6. The photoresist stripping agent according to claim 5, wherein the organic solvent is at least one solvent selected from the group consisting of ether solvents, amide solvents, alcohol solvents, sulfoxide solvents, sulfone solvents, imidazolidinone solvents, and lactone solvents.

7. The photoresist stripping agent according to claim 1, further comprising an anticorrosion agent.

8. The photoresist stripping agent according to claim 7, wherein the anticorrosion agent is at least one compound selected from the group consisting of aromatic hydroxy compounds, sugar alcohols, triazole compounds and chelating compounds.

9. The photoresist stripping agent according to claim 1, further comprising water.

10. The photoresist stripping agent according to claim 1, comprising 0.001 to 100% by weight of the reaction product of formaldehyde and the alkanolamine, and at least one optional component selected from the group consisting of 0 to 99.999% by weight of the alkali compound, 0 to 99% by weight of the organic solvent, 0.1 to 30% by weight of the anticorrosion agent, and 1 to 50% by weight of water, each percentage being selected from respective range so that a total thereof adds up to 100% by weight.

11. The photoresist stripping agent according to claim 1, wherein the reaction product of formaldehyde and the alkanolamine is a formaldehyde-monoethanolamine condensate or a formaldehyde-isopropanolamine condensate.

12. The photoresist stripping agent according to claim 1, wherein the reaction product of formaldehyde and the alkanolamine is produced by a method comprising:
a step of slowly adding formaldehyde to a predetermined amount of the alkanolamine over 30 to 1200 min under stirring while maintaining a temperature of a reaction solution at 70° C. or lower; and
an optional step of further stirring the reaction solution for 30 to 1200 min while maintaining a temperature of the reaction solution at 70° C. or lower,
each of the steps being conducted in an inert gas atmosphere.

13. The photoresist stripping agent according to claim 1, wherein the reaction product of formaldehyde and the alkanolamine shows peaks at least at 45 to 50, 61 to 62 and 64 to 70 ppm when measured by ¹³C-NMR (DMSO-d6).
